(19) United States
(12) Patent Application Publication
(10) Pub. No.: US 2024/0013905 A1
(43) Pub. Date: Jan. 11, 2024

(54) CONNECTIONLESS DATA ALIGNMENT

(71) Applicant: KONINKLIJKE PHILIPS N.V., EINDHOVEN (NL)

(72) Inventors: Jörn BORGERT, HAMBURG (DE); Thomas NETSCH, HAMBURG (DE); Thomas Erik AMTHOR, HAMBURG (DE)

(21) Appl. No.: 18/015,994

(22) PCT Filed: Jul. 15, 2021
(86) PCT No.: PCT/EP2021/069754

§ 371 Date: Jan. 13, 2023

Related U.S. Application Data
(60) Provisional application No. 63/052,489, filed on Jul. 16, 2020.

(51) Int. Cl. G16H 40/63; G16H 10/60; G06K 19/06; G06K 7/14

(52) U.S. Cl. CPC G16H 40/63; G16H 10/60; G06K 19/06037; G06K 7/1417

(57) ABSTRACT

A method (100) for offline entry of execution information to be used by an associated execution device (14) in executing a task includes: providing a user interface (UI) (24) for receiving execution information via the at least one user input device of the defining device and for storing the execution information on the defining device or on an associated data storage accessed by the defining device via an electronic network; constructing at least one graphical pattern (42) encoding the execution information; receiving a trigger input to transfer the stored execution information to the associated execution device; and after receiving the trigger input, displaying the at least one graphical pattern encoding the execution information on the display of the defining device.

The following relates generally to the wireless data transfer arts, data transmission arts, data security arts, medical imaging arts, and related arts.

### BACKGROUND

The situation of having a defining device that defines information for execution at a different execution device is commonplace, as it allows the defining of the information to be done offline and away from the execution device. For example, in medical imaging, radiologist may wish to define a series of imaging scans for a specific patient on a tablet or notebook computer or a cellphone, which are to be later executed by the controller of a medical imaging device. In another example, a person may wish to define a travel itinerary for a train trip on a tablet or notebook computer or a cellphone, which is to be later executed by the scheduling system of a railroad so as to generate and complete the purchase of the appropriate train tickets. As yet another example, a user may wish to preplan the configuration of a computer intended for purchase on a tablet or notebook computer or a cellphone, which is to be later executed by the purchasing system of a computer or electronics retailer so as to generate and complete the computer purchase.

In each of these cases, an issue can arise when the user transfers the information defined on the defining device to the execution device. If a physical cable is used, then the defining device and the execution device must have compatible physical connector ports, which may not be the case. Furthermore, the physical connection may in some instances present a security concern, as the physical connection could potentially be used to transfer malware from one device to the other. The other common approach is to use an electronic network connection such as the Internet. Here, the user must establish an authorized network connection between the defining device and the execution device, usually by providing login information (username and password) to the execution device, which may can raise an issue if the user forgets this login information or does not have an account already created at the execution device. Furthermore, the network connection once established could potentially be used to transfer malware from one device to the other.

The following discloses certain improvements to overcome these problems and others.

### SUMMARY

In one aspect, a non-transitory computer readable medium stores instructions executable by a defining device having an electronic processor, a display, and at least one user input device to cause the defining device to perform a method for offline entry of execution information to be used by an associated execution device in executing a task. The method includes: providing a user interface (UI) for receiving execution information via the at least one user input device of the defining device and for storing the execution information on the defining device or on an associated data storage accessed by the defining device via an electronic network; constructing at least one graphical pattern encoding the execution information; receiving a trigger input to transfer the stored execution information to the associated execution device; and after receiving the trigger input, displaying the at least one graphical pattern encoding the execution information on the display of the defining device.

In another aspect, an apparatus includes a medical imaging device configured to acquire medical images, and a camera. A medical imaging device controller is operatively connected to control the medical imaging device and to configure the medical imaging device to execute a medical imaging task by: receiving, via one or more images acquired by the camera, at least one graphical pattern displayed by an associated defining device; extracting execution information from the at least one graphical pattern; and configuring the medical imaging device to execute the medical imaging task in accordance with the extracted execution information.

In another aspect, a connectionless data transfer method includes: receiving, via at least one user input of a defining device, execution information; generating, at a defining device, a graphical or acoustic representation of the execution information; displaying the graphical acoustic representation via a display of the defining device or transmitting the acoustic representation via a loudspeaker of the defining device; and, at an execution device: imaging the displayed graphical representation with a camera of the execution device or recording the acoustic representation using a microphone of the execution device, extracting the execution information from the imaged graphical representation or the recorded acoustic representation; and executing a task via the execution device in accord with the extracted execution information.

One advantage resides in data transmission between two devices without a physical cable.

Another advantage resides in data transmission between two devices without using a physical cable or a connection to an electronic network.

Another advantage resides in providing for data transmission between two devices without a risk (or, at least, with reduced risk) of transmission of malicious software

Another advantage resides in providing for transmitting data to an execution device to execute instructions in the data without manually having to enter data into the execution device.

Another advantage resides in providing for transmitting execution information from a defining device to an execution device in a way that requires a line-of-sight between the two devices, but which does not use a physical cable or physical network connection.

Another advantage resides in providing for secure transmission of execution information from a defining device to an execution device using hardware commonly included in such devices such as a display or a built-in webcam.

Another advantage resides in providing for secure transmission of execution information from a defining device to an execution device in environments such as a magnetic resonance imaging laboratory that are not amenable to use of wireless electronic networks.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for secure transmission of execution information (e.g. imaging device configuration data) from a mobile device (e.g. cellphone) to an execution device (e.g. imaging device controller).

FIG. 2 shows an example flow chart of secure data transfer operations suitably performed by the apparatus of FIG. 1.

FIG. 3 diagrammatically shows a series of user interface displays presented on the devices of the apparatus of FIG. 1 during performance of the method of FIG. 2.

### DETAILED DESCRIPTION

The following discloses systems and methods for connectionless data transmission by leveraging a displayed visual pattern, such as a matrix barcode, to visually transfer the information generated at the defining device to the execution device. In the illustrative embodiments, a two-dimensional Quick Response (QR) code and/or a one-dimensional Universal Product Code (UPC) barcode is used as the visual pattern.

To implement this solution, the defining device is programmed to store the information generated at the defining device at a non-transitory data storage of (or accessible by) the defining device. The defining device further includes a display. When the user wants to transfer the information to the execution device, the defining device is further programmed to retrieve the stored information from the non-transitory data storage, generate a QR code (or other spatial pattern) encoding the retrieved information in accordance with the OR encoding scheme standard, and display the generated QR code on the display of the defining device.

The execution device is equipped with a camera capable of capturing an image of the QR code displayed on the display of the defining device, and is programmed to decode the imaged QR code using the standard QR decoding scheme.

Advantageously, the data transfer requires a line-of-sight between the display of the defining device and the camera of the execution device, which limits the likelihood of intercepting the communication link or misusing the communication link with malicious intent. The generated QR code is only displayed briefly (and optionally is only created in response to the user selecting a “transfer data” option or the like) and is thereafter optionally destroyed (both by removing the QR code from the display and by deleting the data structure storing the QR code). If the execution device camera shutter speed (i.e., the ability to acquire one image) provides a frame rate that is fast enough, the QR code could even be displayed for only a fraction of a second. In its basic form the data transfer is unidirectional (from the defining device to the execution device) which means there is no way for the execution device to transfer malware to the defining device. Moreover, as long as the execution device is programmed to use the information decoded from the QR code for an intended purpose such as setting MRI scan parameters, the potential for transfer of malware from the defining device to the execution device is also small or nonexistent. A QR code also stores a limited amount of information (7,089 numeric characters or 4,296 alphanumeric characters at low error correction level for version 40, i.e. 40-L, and even less information at higher error correction levels), again limiting the potential for malware transfer. Still further, the approach leverages existing QR encoding/decoding technology (and/or UPC barcode encoding/decoding technology or the like) so that it is straightforward to implement using existing computer technology and an existing webcam or other camera.

In some embodiments disclosed herein, the information contained in the QR code can be encrypted using standard public/private encryption, so that only the execution device can decrypt the information.

In other embodiments disclosed herein, a portion of the information that is transferred in the QR code is also independently stored at (or accessible by) the execution device, thus providing a data check. For example, in the context the execution device being a medical device and the information being configuration information for using the medical device for a specific patient, the information stored at the QR code may include a patient ID which is also available at the execution device (for example, read from a hospital database), and the execution device can thereby verify that the configuration information is indeed for the correct patient.

A bidirectional information transfer is also disclosed. This requires additionally providing a display at the execution device and further programming to encode and display QR codes at the execution device, and a camera at the defining device and further programming to decode the QR code presented by the execution device. If the defining device is a tablet or notebook computer or a cellphone then it likely already has a built-in camera, and a device such as an imaging device controller already has a display. The bidirectional information can be used, for example, to exchange public encryption keys or other authentication information, or to exchange patient ID to further ensure the medical device configuration is for the correct patient. For example, the imaging technician can start scan setup for a patient and then send the full configuration using his/her cellphone, and in response the imaging device controller can send back the patient ID—if this does not match the patient ID stored at the cellphone then a warning alert can be shown on the cellphone. In another example, medical information, such as patient information, can be encrypted using the public encryption keys or other authentication information exchanged as part of the bidirectional information. For example, initially the defining and execution devices can display QR codes for exchanging the public encryption keys, and then thereafter one or both devices can construct and display one or more QR codes conveying medical information encrypted using the exchanged keys. In another approach, the technologist can use data transfer from the imaging device controller to his/her cellphone to retrieve the configurable scan settings for a particular imaging device so that the technologist can configure upcoming scans for that particular imaging device offline using his/her cellphone.

In some embodiments disclosed herein, the defining device can print the generated QR code on a physical piece of paper using a printer or other marking engine. This might be useful if, for example, the execution device is at a location where cellphones are not permitted for security reasons, e.g. a restricted military base, or for practical reasons, such as in a magnet room, or another methodology can be used, such as an intermediary relay device that does not have location restrictions, but also access the executing device.

In other embodiments disclosed herein, the QR code could be transferred from a desktop computer or other stationary defining device to a mobile device such as a cellphone, simply by using the cellphone to take a photograph of the QR code displayed on the desktop computer. The cellphone can then subsequently be used to present the QR code to the execution device by bringing up the QR code photograph on the cellphone display.

As previously noted, the QR code has limited information capacity. This can be increased by data compression in some instances. In another approach, the defining device can be programmed to convey information too large to encode in a single QR code by sequentially encoding and displaying a series of QR codes in rapid sequence (e.g. one QR code displayed per second); and the execution device is then programmed to read and decode the sequence of OR codes to receive the full information.

In some examples, a displayed visual pattern, specifically QR codes and (in one example) a UPC barcode is implemented. In other examples, the visual pattern could be displayed as an infrared image, if the defining device display is capable of this and the camera of the execution device camera range extends into the infrared. A visual pattern employing color encoding is also contemplated, such as employing a High Capacity Colored 2-Dimensional (HCC2D) Code. This can increase informational capacity, but requires both the defining device display and the execution device camera to have accurate color rendering/capture.

In other examples, the visual pattern is replaced by an audio signal played by a loudspeaker of the defining device and received by a microphone of the execution device. For example, the information generated at the defining device can be encoded onto an audio carrier signal using frequency modulation (FM), amplitude modulation (AM), phase shift keying (PSK), or any other suitable audio modulation technique. The carrier signal can be in the acoustic range (usually considered to be 20 Hz to 20 kHz) in which case it will be audible to human bystanders, or in the ultrasonic range (>20 kHz) in which case it will be inaudible to human bystanders but audible to microphone with suitable high-frequency response.

The data transfer could also be performed by way of a low-power wireless electronic communication link, such as an infrared link, Bluetooth link, or the like. However, usually such electronic communication links require some sort of bidirectional communication to establish the link (e.g., Bluetooth pairing) which increases security risk and reduces simplicity. In some preferred embodiments, there is no electronic network link between the defining device and the execution device at the time of data transfer. (Rather, the link is visual, via a displayed QR code or the like, or acoustic).

While described herein primarily in reference to medical imaging, the disclosed systems and methods are applicable in any field in which data transmission is implemented.

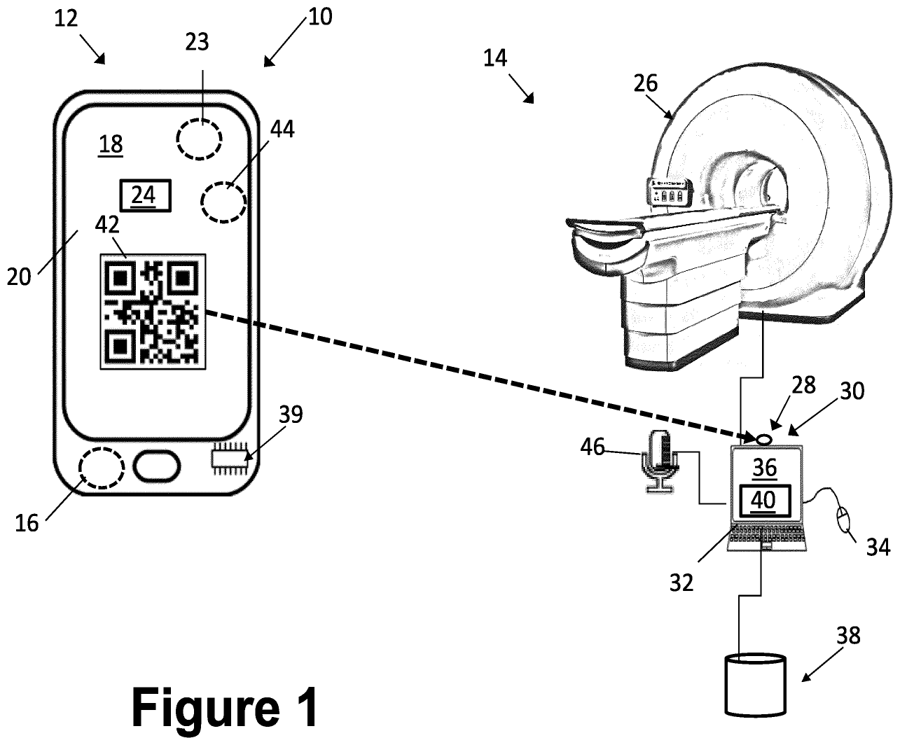

With reference to FIG. 1, a system or apparatus 10 for offline entry of execution information for executing a task is shown. As shown in FIG. 1, the system 10 includes a defining device or apparatus 12 and an execution device or apparatus 14. In some examples, the defining device 12 can be a mobile device (e.g., an illustrative cellular telephone 12, or a tablet computer, personal data assistant or PDA, and/or so forth) operable by a user. The defining device 12 includes typical mobile device components, such as an electronic processor 16, a display 18, and at least one user input device 20 (e.g., a touchscreen to receive user inputs via which the user can swipe with a finger). The defining device 12 also includes a data storage 39 storing instructions for execution information on the defining device to execute a task by the execution device 14. A camera 23 is configured to acquire one or more images.

The defining device 12 is configured to generate a representation for transmission to the execution device 14 to execute a task. The user can use a mobile application program (“app”) 24 which is loaded on, and executable on, the mobile device 12. The app 24 may be downloaded to the mobile device 12 from an app store accessed via a Wi-Fi, cellular, or other wireless communication network. In a suitable embodiment, the app 24 is represented on the home screen or applications screen (e.g., the UI 24) of the mobile device 12 as an app icon (i.e. a small square, round, or other compact graphical element representing the app 24) and the user launches (i.e. initiates running of) an instance of the app 24 on the mobile device 12 by touching the icon on a (touch-sensitive) screen of the mobile device 12.

As shown in FIG. 1, the execution device 14 includes a medical imaging device (or image acquisition device, imaging device, or variants thereof) 26 that in the illustrative example includes a controller 30. The medical imaging device 26 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; a C-arm angiography imager, or a medical imaging device of another modality. The imaging device 2 may also be a hybrid imaging device such as a PET/CT or SPECT/CT imaging system. These are merely examples, and should not be construed as limiting. In addition, as noted, the execution device 14 can be any suitable device for receiving the representation from the defining device 12.

A camera 28 mounted to an exterior of the medical imaging device 26. In the illustrative embodiment, the camera 28 is a webcam 28 installed in a bezel of a display device 36 of the controller 30 of the imaging device. The camera is used to acquire one or more images of the representation from the defining device 12. An imaging technician, or other operator controls the medical imaging device 26 via an imaging device controller 30. As shown in FIG. 1, the medical imaging device controller 30 comprises a workstation, such as an electronic processing device, a workstation computer, or more generally a computer. Additionally or alternatively, the medical imaging device controller 30 can be embodied as a server computer or a plurality of server computers, e.g. interconnected to form a server cluster, cloud computing resource, or so forth. The medical imaging device controller 30 includes typical workstation components, such as an electronic processor 32 (e.g., a microprocessor), at least one user input device (e.g., a mouse, a keyboard, a trackball, and/or the like) 34, and at least one display device 36 (e.g. an LCD display, plasma display, cathode ray tube display, and/or so forth) and the illustrative webcam 28 (alternatively, an external camera could be used that is connected with the controller 30 by a USB cable or the like). In some embodiments, the display device 36 can be a separate component from the medical imaging device controller 30. The display device 36 may also comprise two or more display devices.

The images acquired by the camera 28 that contain the representation are processed to extract the execution information encoded into the representation. The electronic processor 32 of the imaging device 26 (and more particularly of the controller 30 in the illustrative example) is operatively connected with a one or more non-transitory storage media 38. The non-transitory storage media 38 may, by way of non-limiting illustrative example, include one or more of a magnetic disk, RAID, or other magnetic storage medium; a solid state drive, flash drive, an optical disk or other optical storage; various combinations thereof; or so forth; and may be for example a network storage, an internal hard drive of the workstation 30, various combinations thereof, or so forth. It is to be understood that any reference to a non-transitory medium or media 38 herein is to be broadly construed as encompassing a single medium or multiple media of the same or different types. Likewise, the electronic processor 32 may be embodied as a single electronic processor or as two or more electronic processors. The non-transitory storage media 38 stores instructions executable by the at least one electronic processor 32. The instructions include instructions to generate a graphical user interface (GUI) 40 for display on the display device 36.

FIG. 1 also shows an example of a representation 42 generated by the defining device 12 for transmission to the execution device 14. The representation 42 includes instructions executable by the execution device (e.g., the electronic processor 32 of the imaging device controller 30) to execute a task. In some examples, the representation 42 comprises an acoustic transmission transmitted via a loudspeaker 44 of the defining device 12, and received by a microphone 46 of the execution device. In most embodiments, the representation 42 comprises at least one graphical pattern encoding the execution information for the execution device 14 to execute a task. For example, as shown in FIG. 1, the at least one graphical pattern 42 comprises a one-dimensional barcode and/or a two-dimensional matrix barcode. The defining device 12 further includes a non-transitory storage medium 39, for example, a read only memory (ROM), flash memory, electronically erasable programmable read only memory (EEPROM), an SD card, microSD card, or the like, that stores instructions which are readable and executable by the electronic processor 16 of the defining device 12. Note that the non-transitory storage medium 39 is diagrammatically shown in FIG. 1, and is usually an internal component disposed inside the cellphone or other mobile device 12 and hence hidden from view.

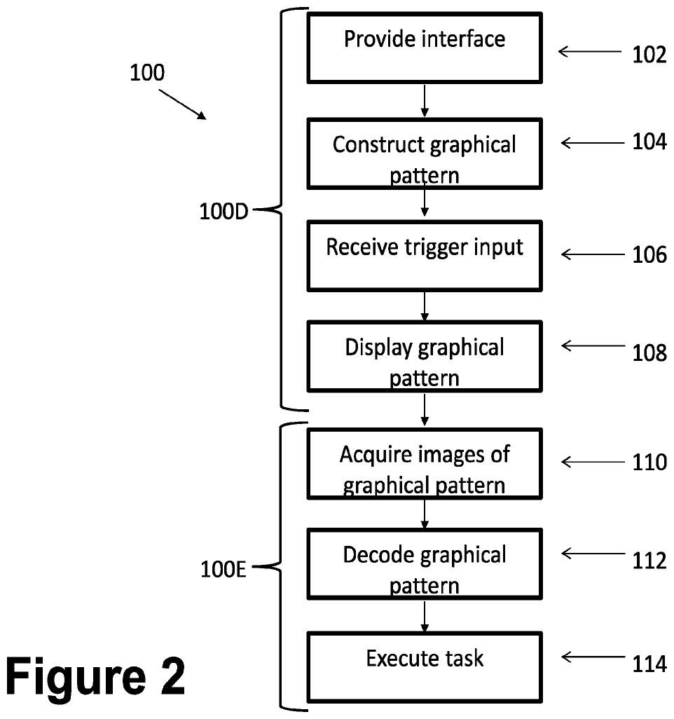

The mobile device 12 and the medical imaging device controller 30 are configured as described above to perform a method or process for offline entry of execution information to be used in executing a task (for example, a method or process 100 shown in FIG. 2). The electronic processor 16 of the defining device 12 reads and executes instructions stored on the non-transitory storage medium 39 of the defining device 12, and the at least one electronic processor 32 (of the medical imaging device controller 10, as shown, and/or the electronic processor or processors of a server or servers on a local area network or the Internet) reads and executes instructions stored on the non-transitory storage medium 38 to perform disclosed operations including performing the method or process 100. In some examples, the method 100 may be performed at least in part by cloud processing.

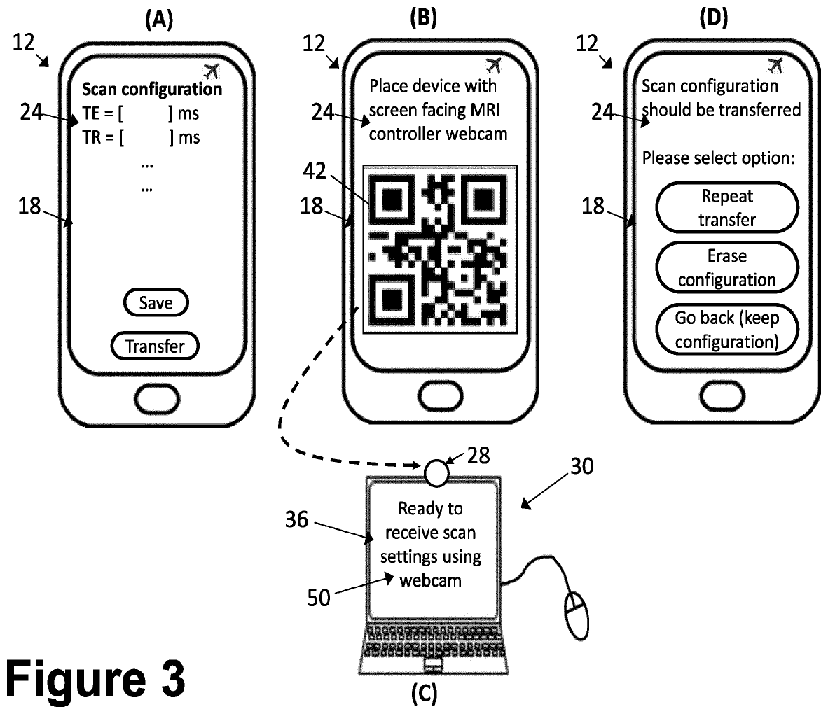

With reference to FIGS. 2 and 3, and with continuing reference to FIG. 1, an illustrative embodiment of the method 100 is diagrammatically shown as a flowchart. A first portion 100D of the method 100 is performed by the defining device 12, and a second portion 100E of the method 100 is performed by the execution device 14. To begin the method 100, the user of the defining device 12 accesses the app 24 (or downloads the app from an associated app store and then accesses the app).

At an operation 102 performed at the defining device 12, a user interface UI, for example, the app 24, is provided on the display device 18 of the defining device 12. The app 24 is configured to receive execution information via the at least one user input device 20 of the defining device 12 (e.g., the user inputting inputs to the app 24 via the touch screen 20 to generate the execution information). The execution information includes information for a task to be performed by the execution device 14, such as a medical imaging examination to be performed by the medical imaging device 26. The execution information can include, for example, scan settings, an anatomy of a patient to be imaged, a number of images to be acquired, and so forth. FIG. 3 Part (A) shows an illustrative UI 24 for entering MRI scan parameters such as time-to-echo (TE), repeat time (TR), and so forth at the mobile device 12. The execution information can be stored in the data storage 39 (see FIG. 1) of the mobile device 12 (or in an associated cloud data storage accessed by the defining device 12 via a Wi-Fi network, 4G or other cellular network or the like), for example by pressing an illustrative “Save” button presented on the UI 24 shown in FIG. 3 Part A. It will be appreciated that the data entry interface can be provided at operation 102 at any location, e.g. while the user of the mobile device 12 is not near to the imaging device 26. For example, operation 102 can be performed while the user is at home, or in a medical office, or so forth. By saving the entered execution information in the data storage 39 of the mobile device 12 (or in a cloud storage linked to the mobile device 12) it follows that the entered execution information is carried with the mobile device 12.

At an operation 104 performed at the defining device 12, at least one graphical (or audio) pattern 42 is constructed to encode the execution information. At an operation 106, a trigger input is received at the defining device 12 to transfer the stored execution information in the graphical pattern 42 to the execution device 14. In some examples, the order of the operations 104 and 106 can be reversed. That is, the user can provide the trigger input on the mobile device 12, and in response the mobile device generates the graphical pattern 42. In some examples, the receiving of the trigger input operation 106 can include providing a data transfer interface on the display 18 of the mobile device 12 for receiving the trigger input via the touchscreen 20 (e.g., via a finger tap or swipe on the touch-screen 20 of the mobile device 12). In other examples, the camera 23 of the mobile device 12 is configured to receive the trigger input as a detection of a position of the mobile device respective to the camera 28 of the execution device 14. That is, the camera 23 uses a pattern recognition process to detect when the mobile device 12 is correctly positioned with respect to the medical imaging device 26. The camera 23 then displays the graphical pattern 42 on the display 18 in an operation 108 to implement the data transfer. FIG. 3 illustrates an example of the operations 106, 108 as manifested on the display 18 of the mobile device 12. In this nonlimiting illustrative example, the UI 24 shown in FIG. 3 Part A further includes a “Transfer” button which, when pressed by the user (it is assumed here that the display 18 is a touch-sensitive display) serves as the trigger signal receipt operation 106. In response to this operation 106, the UI 24 displays the QR code 42 (constructed from the entered execution information at operation 104) on the display 18.

In some variant embodiments, the trigger receipt operation 106 can include capturing an image of a graphical pattern 42 associated with the execution device 14 with the camera 23 of the defining device 12. For example, a sticker or paper including the graphical pattern 42 can be placed on the medical imaging device 26, or the graphical pattern can be displayed on the display device 36 of the medical imaging controller 30. The graphical pattern 42 include identification information about the medical imaging device 26. The graphical pattern 42 associated with the execution device 14 is decoded by the electronic processor 16 of the mobile device 12 to receive information from the associated execution device that serves to trigger the display operation 108. In this embodiment, the trigger input includes the extracted information from the execution device 14. The trigger input in this example could be the correct decrypted information, or it might not be encrypted, (e.g. the graphical pattern 42 associated with the execution device 14 can simply be a barcode encoding a serial number of the medical imaging device 26 or a patient ID (shown on the screen) of a patient to be imaged).

At the operation 108 performed at the defining device 12, the at least one graphical pattern 42 encoding the execution information is displayed on the display 18 of the defining device 12 (e.g., as shown in FIG. 3 Part B). In some examples, for security of the execution information, the graphical pattern 42 is displayed on the display 18 for a less than a predetermined time periods (e.g., 5 seconds or less, one or more tenths of a second, or any other suitable time period). In some embodiments, two or more graphical patterns 42 are generated, and are displayed one after each other in a time sequence.

At an operation 110 performed at the execution device 26 (and, more particularly at the controller 30 in the example of FIG. 3 Part C), the camera 28 of the execution device 14 is configured to acquire one or more images of the graphical pattern 42 from the mobile device 12. In some examples, multiple graphical patterns 42 can be displayed in a time sequence on the mobile device 12, and the camera 28 is configured to acquire images of each displayed graphical pattern 42. In some embodiments, the user performs a setup of the execution device to prepare the execution device to receive the execution information. For example, although not shown in FIG. 3, the user when setting up the imaging device 26 may reach a dialog screen of the MRI controller UI at which the scan setup parameters are to be entered. This dialog screen suitably includes a button or other user input to select to receive the scan setup parameters via a mobile device, and in response a message 50 (see FIG. 3 Part C) is displayed on the display 36 of the imaging device controller 30 instructing: “Ready to receive scan settings using webcam”. This also places the controller 30 into a mode in which it is acquiring video using the webcam 28. The video frames are processed to detect a captured image of the QR code 42 in a video frame, at which point the video frame containing the image of the QR code 42 serves as the acquired image of the graphical pattern 42.

At an operation 112 performed at the execution device 26, the medical imaging device controller 30 is configured to decode and extract the execution from the graphical pattern 42. In some examples, the graphical pattern 42 comprises a two-dimensional matrix barcode (such as the illustrative QR code 42) and the extracting of the execution information comprises decoding the two-dimensional matrix barcode.

At an operation 114 performed at the execution device 26, the medical imaging device controller 30 configures the medical imaging device 26 to execute a medical imaging task in accordance with the extracted execution information. That is, the medical imaging device controller 30 uses the execution information decoded from the graphical pattern 42 to adjust settings of the medical imaging device 26 for an imaging examination. In some examples, a patient identification can be retrieved by the medical imaging device controller 30 from a patient database (e.g., an electronic health or medical record, which is not shown) for a patient who is to be imaged by executing the medical imaging task. The medical imaging device 26 is then configured by comparing a patient identification information component of the execution information with the retrieved patient identification to confirm the execution information is for the medical imaging task being configured. In the example of FIG. 3 the operation 114 would entail configuring the scan settings for the upcoming MRI scan to the scan settings extracted from the QR code 42.

In some examples, the configuring operation 114 includes transmitting a status of the operator of the medical imaging device 26. For example, the medical imaging device controller 30 is configured to construct a graphical pattern encoding information about the medical imaging device 26 and/or about the medical imaging task, which can be displayed on the display device 36 of the controller.

With reference to FIG. 3 and particularly to FIG. 3 Part D, the mobile device 12 preferably has some user control to handle situations such as a failure of the execution device 26 to read the graphical pattern 42, or to handle an accidental pressing of the “Transfer” button in the UI 24 shown in FIG. 3 Part A, or so forth. Such user control is useful since in some embodiments the defining device 12 does not receive feedback from the execution device 26 (indeed, in some embodiments there is no communication at all between the devices 12, 26 other than that provided by the operations 108, 110 of FIG. 2).

In the illustrative example of FIG. 3 Part D, after displaying the graphical pattern 42 for a predetermined time as shown in FIG. 3 PART B (e.g. displaying the QR code 42 for 5 seconds, 10 seconds, or so forth), the UI 24 of the defining device 12 then switches to the dialog shown in FIG. 3 Part D, which provides the user with follow-up selection buttons. A “Repeat transfer” button can be pressed by the user if the camera 28 failed to capture the graphical pattern 42 for some reason (such as, the user failing to hold up the mobile device 12 in front of the webcam 28, or doing so after the QR code has ceased to be displayed). An “Erase configuration” button is provided to allow the user to erase the execution information (e.g. scan settings) that were entered into the mobile device 12 at FIG. 3 Part A. This option would be appropriate if the scan settings were successfully transferred and the user no longer wants to have them stored on the mobile device 12. (Preferably, pressing this button will bring up a confirmation user dialog, not shown, where the user confirms the intent to delete the execution information before it is actually deleted). Finally, a button “Go back (keep configuration)” goes back to the display of FIG. 3 Part A without erasing the execution information. This might be an appropriate option for the user to select if the “Transfer” button in the UI dialog of FIG. 3 Part A were inadvertently selected at some time when the user is not ready to perform the MRI scan, or if the user wishes to retain the execution information (e.g. scan settings) on the defining device 12 for use in future MRI scans.

Typically, the data transmission between the defining device 12 and the execution device 14 comprises a unidirectional transmission (e.g., from the defining to device to the execution device) for security and to prevent transmission of malicious software from the execution device to the mobile device. However, in some embodiments, this transmission can be bi-directional. Communication from the execution device to the defining device can be used in various ways. In one use situation, the display 36 of the controller 30 displays a confirmation that the scan settings were received, and this confirmation is captured by the camera 23 of the defining device 12. This type of confirmation signal could eliminate the need for the follow-up display of FIG. 3 Part D. In another use situation, the controller 30 displays information such as patient ID of the patient who is about to be scanned (possibly encoded in a bar code, QR code, or other graphical representation) and this information is captured by the camera 23 of the defining device 12 and compared with corresponding information (e.g. patient ID) which forms part of the execution information. The defining device 12 can thus verify that it is sending scan settings for the correct patient, or indicate an error if the patient ID received via the camera 23 does not match the patient ID component of the execution information stored at the mobile device 12.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by a defining device having an electronic processor, a display, and at least one user input device to cause the defining device to perform a method for offline entry of execution information to be used by an associated execution device in executing a task, the method comprising:
providing a user interface (UI) for receiving execution information via the at least one user input device of the defining device and for storing the execution information on the defining device or on an associated data storage accessed by the defining device via an electronic network;
constructing at least one graphical pattern encoding the execution information;
receiving a trigger input to transfer the stored execution information to the associated execution device; and
after receiving the trigger input, displaying the at least one graphical pattern encoding the execution information on the display of the defining device.

2. The non-transitory computer readable medium of claim 1, wherein the at least one graphical pattern comprises at least one of a one-dimensional barcode or a two-dimensional matrix barcode.

3. The non-transitory computer readable medium of claim 1, wherein the constructing of the at least one graphical pattern is performed after receiving the trigger input.

4. The non-transitory computer readable medium of claim 1, wherein the at least one graphical pattern is displayed for 5 seconds or less.

5. The non-transitory computer readable medium of claim 1, wherein the at least one graphical pattern includes two or more graphical patterns and the displaying comprises displaying each graphical pattern of the two or more graphical patterns one after another in a time sequence.

6. The non-transitory computer readable medium of claim 1, wherein the receiving of the trigger input comprises at least one of:
providing a data transfer user interface for receiving the trigger input via the at least one user input device of the defining device; and
via a camera of the defining device, receiving the trigger input as a detection of a position of the defining device respective to a camera of the associated execution device.

7. The non-transitory computer readable medium of claim 1, wherein the method further comprises:
via a camera of the defining device, capturing an image of a graphical pattern associated with the execution device; and
decoding the graphical pattern associated with the execution device to receive information from the associated execution device.

8. The non-transitory computer readable medium of claim 7, wherein the trigger input comprises the extracted information from the associated execution device.

9. The non-transitory computer readable medium of claim 7, wherein the information received from the associated execution device by decoding the graphical pattern associated with the execution device includes authentication information and the constructing of the at least one graphical pattern encoding the execution information includes encrypting the execution information using the authentication information.

10. An apparatus, comprising:
a medical imaging device configured to acquire medical images;
a camera; and
a medical imaging device controller operatively connected to control the medical imaging device and to configure the medical imaging device to execute a medical imaging task by:
receiving, via one or more images acquired by the camera, at least one graphical pattern displayed by an associated defining device;
extracting execution information from the at least one graphical pattern; and
configuring the medical imaging device to execute the medical imaging task in accordance with the extracted execution information.

11. The apparatus of claim 10, wherein the at least one graphical pattern comprises a two-dimensional matrix barcode and the extracting of the execution information comprises decoding the two-dimensional matrix barcode.

12. The apparatus of claim 10, wherein the receiving comprises receiving, via a time sequence of images acquired by the camera, a time sequence of graphical patterns displayed by the associated defining device.

13. The apparatus of claim 10, wherein the medical imaging device controller includes a display, and is configured to:
construct a graphical pattern encoding information about the medical imaging device and/or about the medical imaging task; and
displaying the graphical pattern on the display of the medical imaging device controller.

14. The apparatus of claim 10, wherein the medical imaging device controller configures the medical imaging device to execute the medical imaging task by further:
retrieving a patient identification from an electronic database for a patient who is to be imaged by executing the medical imaging task; and
comparing a patient identification information component of the execution information with the retrieved patient identification to confirm the execution information is for the medical imaging task being configured.

15. A system comprising:
the apparatus of claim 10; and
a defining device having a display and at least one user input device and configured to:
provide a user interface (UI) for receiving the execution information via the at least one user input device of the defining device;
construct the at least one graphical pattern encoding the execution information; and
display the at least one graphical pattern encoding the execution information on the display of the defining device.

16. A connectionless data transfer method comprising:
receiving, via at least one user input of a defining device, execution information;
generating, at a defining device, a graphical or acoustic representation of the execution information;
displaying the graphical acoustic representation via a display of the defining device or transmitting the acoustic representation via a loudspeaker of the defining device; and
at an execution device:
imaging the displayed graphical representation with a camera of the execution device or recording the acoustic representation using a microphone of the execution device;
extracting the execution information from the imaged graphical representation or the recorded acoustic representation; and
executing a task via the execution device in accord with the extracted execution information.

17. The method of claim 16, wherein the graphical representation comprises at least one graphical pattern comprises at least one of a one-dimensional barcode or a two-dimensional matrix barcode.

18. The method of claim 16, wherein the at least one graphical pattern includes two or more graphical patterns and the displaying comprises displaying each graphical pattern of the two or more graphical patterns one after another in a time sequence.

19. The method of claim 16, further including at least one of:
providing a data transfer user interface for receiving a trigger input via the at least one user input device of the defining device; and
via a camera of the defining device, receiving a trigger input as a detection of a position of the defining device respective to a camera of the associated execution device.

20. The method of claim 15, wherein the representation comprises an acoustic representation that is transmitted via the loudspeaker of the defining device.
